(19) United States
(12) Patent Application Publication
(10) Pub. No.: US 2006/0193922 A1
(43) Pub. Date: Aug. 31, 2006

(54) Compositions and methods for the treatment of acne

(76) Inventor: Natalia Neikrug, Beverly Hills, CA (US)

Correspondence Address: JONES DAY, 555 SOUTH FLOWER STREET FIFTIETH FLOOR, LOS ANGELES, CA, 90071, US

(21) Appl. No.: 11/066,110

(22) Filed: Feb. 25, 2005

(51) Int. Cl. A61K 36/00; A61K 35/55

(52) U.S. Cl. 424/563; 424/725; 424/400

(57) ABSTRACT

This invention relates to compositions for the treatment of acne. In a preferred embodiment, the composition comprises adrenal extract and bupleurum root. In another preferred embodiment, the composition further includes licorice root. In another preferred embodiment, the invention also includes at least one vitamin B5 component, a magnesium component, a zinc component and an amino acid component. The invention also relates to methods for treating acne by administering the compositions in an amount therapeutically effective in resolving acne lesions and preventing their reappearance.

### FIELD OF THE INVENTION

The present invention relates to compositions for the treatment of acne. The present invention further relates to methods for the treatment of acne by administering the compositions of the present invention to the subject afflicted with acne.

### BACKGROUND OF THE INVENTION

Acne vulgaris, also called acne, pimple, or break out, is the most common disease of the pilosebaceous unit of the skin. It is estimated that 30 percent of teenagers have acne of sufficient severity to require medical treatment. Acne affects 25 percent of all adult men and 50 percent of adult women sometimes in their adult lives. People can develop acne, or have a recurrence of acne, in their 30s, 40s, and sometimes beyond that. Acne is most commonly distributed on the face and to a lesser degree on the back and chest. It can persist for years and result in disfigurement and permanent scarring of the skin. The past few decades have seen very little progress toward the development of medicines with potent efficacy and low toxicity to treat acne.

Acne conglobate, more commonly known as nodular or cystic acne, is a more severe form of acne than acne vulgaris. In the case of nodular acne, the sebum builds up in the gland, mixes with dead cells, and eventually ruptures the follicle wall, which typically forms a deep cyst under the skin. Scarring often results from these deep cysts. (Roche Laboratories Inc., Important Information Concerning Your Treatment with Accutane, 6^{th} ed., 1996).

Androgen is one of the most important factors to cause an increased production of sebum. From the age of puberty period and up, the adrenal glands mature and secrete an increased amount of androgen to increase the activity of sebaceous glands and produce more sebum. Males produce 10 times as much androgen as females, so it is not surprising that more males develop severe cases of acne.

With a few dozen acne medications out on the market, it is difficult for consumers to find a good product that really works for them because there are only a few that can actually treat acne. Most products contain basically the same ingredients: retinoid and its derivatives, benzyl peroxide, salicylic acid, sulfur, antibiotics, etc. Most are only effective for a short period of time and posses many side effects.

Most of the over the counter medications contain salicylic acid, sulfur, benzyl peroxide, etc. Most can only treat the minor aspect of acne. These topical creams, lotions, ointments and cleansers can only attack the end result of acne, but not the causes. They cannot prevent future outbreaks.

The prescribed topical cream and other formulations, like retin-A and its derivatives and antibiotics (benzamycin), may be able to treat part of the acne problem, but the side reactions which they cause often outweigh the benefits. They strip off layers of the skin, dry and irritate skin, and make skin more susceptible to sun burns.

The other current treatment is systemic treatment, such as antibiotics. About two thirds of acne sufferers respond to this treatment. However, treatment with antibiotics is a long-term treatment that causes many side reactions, as well as destroying the balance of the body's bacterial system, which will create additional problems.

The only treatment that is proven effective in the vast majority of cases is the oral administration of isotretinoin (Accutane™). This medication, however, has numerous side effects; the most disturbing of which being its potential to induce severe birth defects.

Although acne is not a systemic disease and is not in and of itself life threatening, it causes substantial emotional discomfort. Virtually all acne sufferers are willing to try any therapy that they think will be effective. Therefore, there exists a clear need in the art for safe non-toxic compositions and methods that will treat acne without undesirable side effects.

### SUMMARY OF THE INVENTION

The present invention relates to compositions and methods for the treatment of acne comprising mammalian adrenal extracts. The adrenal extract may be in the form of whole adrenal extract, or it may be in the form of adrenal cortical extract. In a preferred embodiment of the present invention, the adrenal extract comprises both whole adrenal extract and adrenal cortex extract. In preferred embodiments, the compositions of the present invention further comprise bupleurum root. In yet other preferred embodiments, the compositions of the present invention further comprise licorice root. The compositions of the present invention may further comprise a component selected from the group consisting of a vitamin B5 component, an amino acid component, a magnesium component, and a zinc component. In preferred embodiments of the present invention, the vitamin B5 component is calcium pantothenate. In preferred embodiments of the present invention, the amino acid component is L-lysine. In preferred embodiments of the present invention, the magnesium component is magnesium chloride. In preferred embodiments of the present invention, the zinc component is a zinc citrate complex.

In another preferred embodiment, the present invention provides compositions for the treatment of acne, comprising whole adrenal extract, ranging from 0.0001-100 percent by weight; adrenal cortex extract, ranging from 0.0001-100 percent by weight; bupleurum root, ranging from 1-99 percent by weight; licorice root, ranging from 1-99 percent by weight; calcium pantothenate, ranging from 1.0-99 percent by weight; L-lysine, ranging from 1-99 percent by weight; magnesium chloride, ranging from 1-99 percent by weight; and zinc citrate, ranging from 1-55 percent by weight.

In yet another preferred embodiment of the compositions for the treatment of acne according to present invention, the whole adrenal extract comprises about 1 to 20 percent by weight; the adrenal cortex extract comprises about 1 to 10 percent by weight; the bupleurum root comprises about 10 to 50 percent by weight; the licorice root comprises about 5 to 25 percent by weight; the calcium pantothenate comprises about 1 to 20 percent by weight; the L-lysine comprises about 5 to 25 percent by weight; the magnesium chloride comprises about 0.5 to 5 percent by weight; and the zinc citrate comprises about 1 percent by weight.

In yet another preferred embodiment of the compositions for the treatment of acne according to present invention, the whole adrenal extract comprises about 10.0 percent by weight; the adrenal cortex extract comprises about 5.4 percent by weight; the bupleurum root comprises about 33.4 percent by weight; the licorice root comprises about 15.1 percent by weight; the calcium pantothenate comprises about 10.0 percent by weight; the L-lysine comprises about 16.7 percent by weight; the magnesium chloride comprises about 8.4 percent by weight; and the zinc citrate comprises about 1.0 percent by weight. The compositions of the present invention may further comprise a pharmaceutically acceptable carrier or excipient.

The present invention also provides method for the treatment of acne comprising administering to a subject afflicted with acne the compositions of the present invention. In a preferred embodiment, the compositions of the present invention are administered orally. In preferred embodiments, the compositions of the present invention are administered in the form of a number of tablets or capsules, preferably about 4, whereby two tablets or capsules are taken in the morning, and two tablets or capsules are taken in the evening. In preferred embodiments, each tablet or capsule comprises about 500 mg to 2.5 g of composition. In more preferred embodiments, each tablet or capsule comprises about 1.5 g of composition. The duration of administration may vary between individuals but is estimated to extend approximately 12 weeks before the acne has been permanently resolved. Significant improvement in the number of lesions and their severity, however, is expected to be noticeable after only two to three weeks of treatment with the compositions and according to the methods of the present invention.

### DETAILED DESCRIPTION OF THE INVENTION

The present invention is based on the discovery that compositions comprising certain combinations of nutrients are able to effectively resolve acne lesions in even the most severe cases of acne. It was surprisingly discovered that compositions comprising adrenal extracts are effective in treating acne within weeks when taken orally. Thus, in preferred embodiments, the compositions of the present invention comprise mammalian adrenal extracts in an amount sufficient to resolve the acne lesions. By adrenal extracts is meant adrenal cortical extracts, adrenal medulla extracts, or a combination of both, such as whole adrenal extracts. Such extracts are readily available in raw, processed, liquid, lyophilized, powder, or any other form suitable for administration in the compositions and methods of the instant invention. Adrenal extracts, including whole adrenal and adrenal cortical extracts, are highly useful in a variety of different modalities. Many commercially available adrenal extracts are derived from the adrenal glands of bovine (beef) sources. Adrenal extracts may also be prepared from porcine, sheep, goat, and human adrenal tissues. However, for the purposes of the present invention, any mammalian-derived adrenal extracts are suitable. The adrenal glands are small lima bean-shaped glands that lie just above the kidneys.

Whole adrenal extract and adrenal cortical extract have previously been demonstrated to be effective in reconstituting adrenal function in cases of adrenal exhaustion, presenting as fatigue, inability to cope with stress and reduced resistance to infection. The importance of adrenal extract preparations in stimulating adrenal and immune functions was first suggested by studies in adrenalectomized animals. It became clear that adrenal gland extracts would prolong their lives. Rats treated with a total extract of the adrenal cortex responded better to stress than animals treated with synthetic corticosteroid mixture (Bernardini R., De Ambrosi L., Pharmacodynamic properties of adrenal cortical extracts in comparison to synthetic corticosteroid mixture in the rat, 1985, Arch. Int. Pharmacodyn. Ther., 276(2), p.287-300. In another study performed on anesthesized dogs, administration of corticoadrenal extract completely abolished the slowing down of respiratory rate as well as the depression of the total peripheral and elastic resistance of arteries usually encountered with anesthesia. Those results suggest that corticoadrenal extract protects anesthetized dogs against the hypotension induced by a sudden fall of blood pressure (Chahine R., et.al., Effects of corticoadrenal extract on haemorrhagic shock-induced perturbation of systemic haemodynamics and general metabolism in anesthesized dogs, 1990, Arzneimittelforschung, 40 (6), p. 654-656). Adrenal Cortical Extract (ACE) readily abolishes the severe symptoms of adrenal insufficiency when allowed to develop and concurrently restores the blood sugar and non-protein nitrogen levels to normal in adrenalectomized cats (Science, Vol. 74, No. 1922, Oct. 30, 1931, pp. 440-441).

Clinical studies in humans using adrenal extracts (adrenal cortical extract and whole adrenal extract) have shown effectiveness in treatments for a variety of conditions that involve low adrenal function, asthma, colds, burns, hypotension, tuberculosis, vomiting during pregnancy, infections, infectious diseases (Harrower H. R., An Endocrine Handbook, 1939, The Harrower Laboratory Inc., p. 19-22). Adrenal extracts have also been used in immunology (Dry J., Leynadier F., Abuaf N., Luce H., Effects in men of a total adrenal cortical extract on delayed immunity and lymphocyte subpopulations, 1978, C. R. Acad. Sci. Hebd. Seances Acad. Sci. D, 286 (21), p. 1543-1545); in obstetrics and gynecology in the prevention of operative stress (Masin G., Piccolomini A., Soma V., Natural adrenal cortex extract in the prevention of operative stress in obstetrics and gynecology, 1981, Clin. Ter., 99(2), p.131-137 ); in geriatric patients (Mazzi C., et al., Extracts of adrenal cortex and their relation with the function of the pituitary-adrenal axis and blood glucose regulation in elderly subjects, 1980, Clin. Ter. 95 (5), p.523-542), (Iori E., Bianchini D., Evaluation of the pharmaco-clinical activity of an association of corticoadrenal extracts, group B vitamins and folinic acid in geriatric patients, 1984, Clin. Ter. 109 (2), p. 137-141); in pneumology (Moreo G., Casalecchi M., Secco G., Clinical experience with a natural high-titer cortical extract in the field of pneumology, 1982, Clin. Ter., 101(3), p. 239-256), (More G., Migliaccio A., Giordano G., High-titer adrenal cortex extract as collateral therapy in pulmonary tuberculosis, 1982, Clin. Ter., 100(3), p. 249-259). These studies indicate that adrenal extracts are useful in reconstituting adrenal and immune functions and improving clinical status in these patients.

It has now been surprisingly been discovered that adrenal extracts can be effective in the treatment of acne, particularly when formulated into the compositions and used in the methods of the present invention. Thus, the present invention provides for the first time compositions effective for the treatment of acne comprising adrenal extract. Without wishing to be bound by any particular theory, it is thought that the adrenal extracts used in the compositions and methods of the instant invention not only enhance the healing of existing acne skin lesions but prevent or decrease the infection that occurs in conjunction with acne skin lesions and act to prevent the formation of new lesions.

Adrenal extracts (whole adrenal and adrenal cortex) are usually obtained from pasture-fed, pesticide-free bovine herds. They are typically freeze- dried, vacuum dried extracts. Raw adrenal concentrates are made from toxin-free animal's adrenal glands processed by low lyophilization by sublimation to ensure rawness and preserve natural constituents. They can also be freeze-dried, or never frozen. The liquid form of adrenal extracts usually contains Adrenalinum Sacrode (adrenal gland) 8X or sublingual adrenal extract (whole extract or cortex extract). The adrenal extracts have been completely characterized and are noted for their ability to stimulate adrenal and immune functions, enhance wound healing and decrease microbial adherence.

Commercially available adrenal extracts, such as whole adrenal extract and adrenal cortical extract, also known as Protomorphogen (adrenal), Suprarenal extract, Adrenal raw concentrate are made using the whole gland (whole total adrenal extract), or just the cortex or outer portion of the gland (adrenal cortex extract). They can be found in many different forms including liquid, powder, tablets, capsules, or by injection. Methods of obtaining adrenal extracts, including whole adrenal extracts and adrenal cortical extracts are well known in the art. The four most widely used methods of processing are: the azeotrophic method, salt precipitation, freeze-drying, and predigestion. For purposes of the present invention, any of these methods, as well as others, is suitable for the preparation of the adrenal extracts for use in the compositions and methods of the present invention.

The azeotrophic method begins by quick-freezing the material at well below 0 degrees and then washing the material with a powerful solvent (ethylene dichloride) to remove the fatty tissue. The solvent is then distilled off and the material is dried and ground into a powder so that it can be placed in tablets or capsules. Although the azeotrophic method aids in the removal of fat-stored toxins (like pesticides) and toxic heavy metals, it also removes fat-soluble hormones, enzymes, essential fatty acids, and other potentially beneficial materials.

The salt precipitation method involves the maceration of fresh glandular material in a salt and water solution. Like the azeotrophic method, this process also allows the fat-soluble material to be separated out. The benefit of the salt precipitation method is that no toxic solvents are used to remove the fatty material. The down side is that the salt convent can be very high, and that some of the potentially beneficial constituents may be removed.

The freeze-drying process involves quickly freezing the glandular material at temperatures 40 to 60 degrees below 0 degrees F. and then placing the material into a vacuum chamber, which removes the water by direct vaporization from its frozen state- hence the term freeze-drying. The benefits of freeze-drying are that it preserves more of the unaltered protein and enzyme content of the gland, as well as all of the fat-soluble components. Since the fat is not removed, potentially harmful contaminants that accumulate in fat tissue may remain in the product. It is therefore preferred that the glands be derived from livestock that have grazed on open ranges that are not sprayed with pesticides or herbicides. The animals should also be free of antibiotics, synthetic hormones, and infection.

The predigestion method employs the aid of the plant and animal enzymes to partially digest or hydrolyze the glandular material. The partially digested material is then passed through a series of filtrations to separate out fat-soluble and large molecules. The purified material is then freeze-dried.

In a preferred embodiment of the present invention, the compositions for the treatment of acne comprise a combination of whole adrenal extract and adrenal cortex extract. In preferred embodiments, the ratio of whole adrenal extract and adrenal cortical extract is approximately 2:1 by weight, but the invention is not so limited. It is contemplated that all ratios and weights disclosed herein may be modified and adjusted in accordance with the particular method of manufacture of the compounds. Such adjustments and modifications are well within the skill of the ordinary artisan. Thus, in a preferred embodiment, a daily dose of approximately 600 mg of whole adrenal extract and 320 mg of adrenal cortical extract is contemplated.

In preferred embodiments, the compositions of the present invention further comprise Bupleurum Chinense (root). The active components of Bupleurum are compounds known as saikosaponins. Bupleurum has antibacterial, antiviral, anti-inflammatory and antiseptic properties, which make it highly useful in the treatment of acne in combination with the adrenal extracts according to the compositions and methods of the present invention. In preferred embodiments, a daily dose of approximately 2 grams of bupleurum root is contemplated for the purposes of the present invention. It should be noted, however, that the specific weights described herein are intended merely as guidelines, and not to be construed as limiting the present invention. A person of skill in the art will readily be able to vary these guidelines in accordance with the method of preparation of the ingredients.

In yet other preferred embodiments, the compositions of the present invention comprise licorice root (Glycyrrhiza Glabra). The active component in licorice root is glycyrrhizin. Licorice root also has antibiotic, anti-inflammatory and anti-allergic effects. Without wishing to be bound by theory, it is thought that in the compositions and methods of the present invention licorice root enhances the effect of bupleurum root. In preferred embodiments, the amount of licorice root in the compositions of the present invention comprises a daily dose of approximately 900 mg.

In further preferred embodiments, the compositions of the present invention comprise pantothenic acid. Pantothenic acid, or vitamin B5, is a essential nutrient that helps to reduce allergic reactions and is vital for normal sebum production by the oil glands of the skin. Therefore it is a highly useful component for the compositions of the present invention and it is contemplated at a daily dose of approximately 600 mg, when in the form of calcium pantothenate.

In other preferred embodiments, the compositions of the present invention comprise magnesium. Magnesium is a major mineral involved in the reaction of more than 300 enzymes. It is also necessary for the detoxication of the body. For purposes of the present invention, the magnesium component may be any magnesium compound, but preferably is a magnesium chloride complex. The daily dose of magnesium chloride for the purposes of the present invention is approximately 500 mg.

In yet other preferred embodiments, the compositions of the present invention comprise L-lysine. L-lysine, an essential amino acid, is necessary for tissue repair and the production of antibodies, hormones and enzymes and enhances immune function. The amount of L-lysine contemplated for the purposes of the present invention is approximately 1 gram daily.

In other preferred embodiments, the compositions of the present invention comprise zinc. The zinc component of the compositions of the present invention reduces the inflammation associated with acne. The ability of zinc to promote normal tissue growth, wound repair, collagen formation, and proper immune responses, makes it an effective component of the composition and for the treatment of acne according to the invention. The zinc component may be any zinc compound, but preferably is a zinc citrate complex. In preferred embodiments, the amount of zinc citrate present in the compositions of the present invention comprises approximately 60 mg as a daily dose.

In preferred embodiments, the compositions of the present invention comprise all of the above-mentioned ingredients at the approximate formulation disclosed in Table 1. The compositions may further comprise a pharmaceutically acceptable carrier or excipient. Thus, in another preferred embodiment, the present invention provides compositions for the treatment of acne, comprising whole adrenal extract, ranging from 0.0001-100 percent by weight; adrenal cortex extract, ranging from 0.0001-100 percent by weight; bupleurum root, ranging from 1-99 percent by weight; licorice root, ranging from 1-99 percent by weight; calcium pantothenate, ranging from 1.0-99 percent by weight; L-lysine, ranging from 1-99 percent by weight; magnesium chloride, ranging from 1-99 percent by weight; and zinc citrate, ranging from 1-55 percent by weight.

In yet another preferred embodiment of the compositions for the treatment of acne according to present invention, the whole adrenal extract comprises about 1 to 20 percent by weight; the adrenal cortex extract comprises about 1 to 10 percent by weight; the bupleurum root comprises about 10 to 50 percent by weight; the licorice root comprises about 5 to 25 percent by weight; the calcium pantothenate comprises about 1 to 20 percent by weight; the L-lysine comprises about 5 to 25 percent by weight; the magnesium chloride comprises about 0.5 to 5 percent by weight; and the zinc citrate comprises about 1 percent by weight.

In yet another preferred embodiment of the compositions for the treatment of acne according to present invention, the whole adrenal extract comprises about 10.0 percent by weight; the adrenal cortex extract comprises about 5.4 percent by weight; the bupleurum root comprises about 33.4 percent by weight; the licorice root comprises about 15.1 percent by weight; the calcium pantothenate comprises about 10.0 percent by weight; the L-lysine comprises about 16.7 percent by weight; the magnesium chloride comprises about 8.4 percent by weight; and the zinc citrate comprises about 1.0 percent by weight. The compositions of the present invention may further comprise a pharmaceutically acceptable carrier or excipient.

The present invention also provides method for the treatment of acne comprising administering to a subject afflicted with acne the compositions of the present invention. In a preferred embodiment, the compositions of the present invention are administered orally. In preferred embodiments, the compositions of the present invention are administered in the form of a number of tablets or capsules, preferably about 4, whereby two tablets or capsules are taken in the morning, and two tablets or capsules are taken in the evening. In preferred embodiments, each tablet or capsule comprises about 500 mg to 2.5 g of composition. In more preferred embodiments, each tablet or capsule comprises about 1.5 g of composition. The duration of administration may vary between individuals but is estimated to extend approximately 12 weeks before the acne has permanently resolved. Significant improvement in the number of lesions and their severity, however, is expected to be noticeable after only two to three weeks of treatment with the compositions and according to the methods of the present invention, as further described in the appended examples.

### EXAMPLES

The following examples are provided to better illustrate the claimed invention and are not to be interpreted as limiting the scope of the invention. To the extent that specific quantities are mentioned, it is merely for purposes of illustration and is not intended to limit the invention. It will be understood that many variations can be made in the procedures herein described while still remaining within the bounds of the present invention.

### Example 1

This example illustrates a specific embodiment of the compositions of the present invention. In a preferred embodiment, the present invention provides the following ingredients in the form of a capsule to be ingested daily by the acne sufferer until the disappearance of the acne. The same composition may also be prepared in the form of a pill, tablet, powder or liquid. While the formula as noted is given as a daily dosage, it may be divided into multiple capsules, tablets, or servings.

TABLE 1
Sample formulation for the oral treatment of acne

| Ingredients | Daily Dose | Weight % | Range Percent |
|---|---|---|---|
| Bupleurum root | 2 gm | 33.4 | 1.0-99 |
| Licorice root | 900 mg | 15.1 | 1.0-99 |
| Calcium Pantothenate | 600 mg | 10.0 | 1.0-99 |
| L-Lysine | 1 gm | 16.7 | 1.0-99 |
| Magnesium chloride | 500 mg | 8.4 | 1.0-99 |
| Zinc citrate | 60 mg | 1.0 | 1.0-55 |
| Whole adrenal extract | 600 mg | 10.0 | 0.0001-100 |
| Adrenal cortex extract | 320 mg | 5.4 | 0.0001-100 |

Capsules containing the compositions of the present invention can be prepared by filling standard two-piece hard gelatin capsules each with the desired amount of powdered active ingredients as described above, and may further include inactive ingredients, such as gelatin, starch, or other inactive ingredients known to persons of skill in the art. Similarly, the compositions of the present invention may be formulated as soft gelatin capsules containing the compositions of the present invention in a digestible oil. However, the compositions of the present invention can also be prepared in the form of tablets. Such tablets can be prepared by standard procedures to provide the desired amount of the compositions of the present invention as described in Table 1, and may further comprise appropriate filler, excipient or taste modifying material, as well as appropriate coatings.

### Example 2

This example shows the treatment schedule of a test subject and the effectiveness of the compositions and methods of the present invention in eliminating the subject's acne. The test subject received the composition described in Example 1, above, in a once-daily administration. The following reduction of acne lesions was observed and recorded over time.

Treatment Schedule

| Number of weeks after | |
|---|---|
| beginning of treatment | Number of acne lesions |
| 0 | 41 |
| 2 | 32 |
| 6 | 20 |
| 12 | 0 |

After week 12, the complete absence of acne lesions in the test subject was noted. The treatment was subsequently discontinued and no new acne outbreak was observed within one year. Thus, it can be concluded that the compositions and methods of the present invention are effective in permanently eliminating acne in sufferers without requiring life-long treatment.

What is claimed is:

1. A composition for the treatment of acne comprising mammalian adrenal extract.

2. The composition of claim 1, wherein said adrenal extract comprises both whole adrenal extract and adrenal cortical extract.

3. The composition of claim 2, further comprising bupleurum root.

4. The composition of claim 3, further comprising licorice root.

5. The composition of claim 4, further comprising a component selected from the group consisting of a vitamin B5 component, an amino acid component, a magnesium component, and a zinc component.

6. The composition of claim 5, wherein said vitamin B5 component is calcium pantothenate.

7. The composition of claim 5, wherein said amino acid component is L-lysine.

8. The composition of claim 5, wherein said magnesium component is magnesium chloride.

9. The composition of claim 5, wherein said zinc component is a zinc citrate complex.

10. A composition for the treatment of acne, comprising:
a) Whole Adrenal Extract, ranging from 0.0001-100 percent by weight;
b) Adrenal Cortex Extract, ranging from 0.0001-100 percent by weight;
c) Bupleurum root, ranging from 1-99 percent by weight;
d) Licorice root, ranging from 1-99 percent by weight;
e) Calcium Pantothenate, ranging from 1.0-99 percent by weight;
f) L-Lysine, ranging from 1-99 percent by weight;
g) Magnesium Chloride, ranging from 1-99 percent by weight;
h) Zinc Citrate, ranging from 1-55 percent by weight.

11. The composition for the treatment of acne according to claim 10 wherein the
i) Whole Adrenal Extract comprises about 1 to 20 percent by weight;
j) Adrenal Cortex Extract comprises about 1 to 10 percent by weight;
k) Bupleurum root comprises about 10 to 50 percent by weight;
l) Licorice root comprises about 5 to 25 percent by weight;
m) Calcium Pantothenate comprises about 1 to 20 percent by weight;
n) L-Lysine comprises about 5 to 25 percent by weight;
o) Magnesium Chloride comprises about 0.5 to 5 percent by weight;
p) Zinc Citrate comprises about 1 percent by weight.

12. The composition for the treatment of acne according to claim 10 wherein the
q) Whole Adrenal Extract comprises about 10.0 percent by weight;
r) Adrenal Cortex Extract comprises about 5.4 percent by weight;
s) Bupleurum root comprises about 33.4 percent by weight;
t) Licorice root comprises about 15.1 percent by weight;
u) Calcium Pantothenate comprises about 10.0 percent by weight;
v) L-Lysine comprises about 16.7 percent by weight;
w) Magnesium Chloride comprises about 8.4 percent by weight;
x) Zinc Citrate comprises about 1.0 percent by weight.

13. The composition of claim 1, further comprising a pharmaceutically acceptable carrier or excipient.

14. A method for the treatment of acne comprising administering to a subject the composition of claim 1.

15. The method according to claim 14 wherein said composition is administered orally.

16. The method according to claim 15, wherein said composition is administered as a plurality of tablets or capsules.

17. The method according to claim 16, wherein said plurality of tablets or capsules is about 4.

18. The method according to claim 16, wherein said tablet or capsule comprises about 500 mg to 2.5 g of composition.

19. The method according to claim 17, wherein said tablet or capsule comprises about 1.5 g of composition.

20. The method according to claim 14 wherein said composition is administered twice daily.
